(19) United States
(12) Patent Application Publication
(10) Pub. No.: US 2016/0331764 A1
(43) Pub. Date: Nov. 17, 2016

(54) COMPOSITION COMPRISING XYLITOL AND CHOLECALCIFEROL FOR TOPICAL TREATMENT OF SKIN AND MUCOUS MEMBRANES

(71) Applicant: RESDEVCO RESEARCH AND DEVELOPMENT CO. LTD., Jerusalem (IL)

(72) Inventor: Shabtay DIKSTEIN, JERUSALEM (IL)

(73) Assignee: RESDEVCO RESEARCH AND DEVELOPMENT CO. LTD., JERUSALEM (IL)

(21) Appl. No.: 15/112,433

(22) PCT Filed: Jan. 25, 2015
(86) PCT No.: PCT/IL2015/050083

§ 371 Date: Jul. 19, 2016

Related U.S. Application Data
(60) Provisional application No. 61/932,262, filed on Jan. 28, 2014.

(51) Int. Cl. A61K 31/593; A61K 8/67; A61K 31/047; A61K 8/34; A61K 9/107; A61K 8/06; A61K 31/573; A61K 8/63; A61K 47/10; A61K 47/32; A61K 8/31; A61K 8/81; A61K 8/86; A61Q 19/00; A61K 9/00

(52) U.S. Cl. CPC A61K 31/593; A61K 9/0014; A61K 8/67; A61K 31/047; A61K 8/345; A61K 9/107; A61K 8/06; A61K 31/573; A61K 8/63; A61K 47/10; A61K 8/34; A61K 47/32; A61K 8/31; A61K 8/347; A61K 8/8147; A61K 8/86; A61K 9/0043; A61Q 19/007; A61K 2800/5922; A61K 2800/591; A61K 2800/594

(57) ABSTRACT

A composition for delivering cholecalciferol to skin and/or mucous membranes via topical application. The composition comprises xylitol and cholecalciferol in emulsion. Optionally, the composition may include additional components such as glycerol, polyethylene glycol, propylene glycol, pharmaceutical agents, stabilizers, acids, bases, buffers, antioxidants, emulsifiers, suspending agents, and preservatives. Methods of preparation of the composition and of using it as a topical treatment for delivery of cholecalciferol to the skin and/or mucous membranes are also disclosed.

### REFERENCE TO RELATED APPLICATIONS

This applications claims priority from U.S. Provisional Patent Application No. 61/932,262, filed 28 Jan. 2014.

### FIELD OF THE INVENTION

This invention relates in general to compositions and methods for delivering vitamin D₃ to the skin and mucous membranes. In particular, it relates to topically applied compositions that contain cholecalciferol.

### BACKGROUND OF THE INVENTION

Vitamin D is well-known as being essential for proper assimilation of calcium and phosphate by the body. While vitamin D is commonly recognized as being necessary for proper bone development, recent studies have shown that the skin requires vitamin D as well in order to stay in good condition, and that vitamin D plays a vital role in building up the structure of the skin.

During the winter, particularly in cold climates, the skin cannot produce sufficient vitamin D to meet its needs, and orally ingested vitamin D (e.g. in the form of cholecalciferol, also known as vitamin D₃) is not efficacious for the skin.

A topically-administered vitamin D₃ composition that can provide sufficient vitamin D₃ to the skin or mucous membranes remains a long-felt, yet unmet need.

### SUMMARY OF THE INVENTION

The invention disclosed herein is designed to meet this long-felt need. Topical compositions comprising xylitol and cholecalciferol (vitamin D₃) for delivery of cholecalciferol to the skin or mucous membranes are disclosed. Both pharmaceutical and cosmetic compositions are contemplated by the inventor as being within the scope of the invention.

It is therefore an object of this invention to disclose a composition for delivering cholecalciferol to skin and/or mucous membranes via topical application, wherein said composition comprises xylitol in water and cholecalciferol in emulsion. In preferred embodiments of the invention, said xylitol in water is in the form of an aqueous solution of xylitol. In some embodiments of the invention, said composition is for topical treatment of skin or mucous membranes in which said skin or mucous membrane is damaged. In some embodiments of the invention, said composition is for topical treatment of skin or mucous membranes in which cell proliferation within said skin or mucous membranes is not in healthy equilibrium with cell differentiation within said skin or mucous membranes.

It is a further object of this invention to disclose a composition as defined in any of the above, wherein said composition comprises an aqueous xylitol solution, and further wherein said aqueous xylitol solution is isotonic.

It is a further object of this invention to disclose a composition as defined in any of the above, wherein said composition comprises between 1% and 15% xylitol (w/w). In preferred embodiments of the invention, said composition comprises between 2% and 15% xylitol (w/w).

It is a further object of this invention to disclose a composition as defined in any of the above, wherein said composition additionally comprises glycerol. In some embodiments of the invention, said composition comprises between 1% and 15% glycerol (w/w). In some preferred embodiments of the invention, said composition comprises between 2% and 15% glycerol (w/w). In some preferred embodiments, said composition, said composition comprises an aqueous solution of glycerol and xylitol in which said aqueous solution of glycerol and xylitol is isotonic.

It is a further object of this invention to disclose a composition for delivering cholecalciferol to skin and/or mucous membranes via topical application, wherein said composition consists of: (a) xylitol; (b) cholecalciferol in emulsion; (c) optionally, at least one substance selected from the group consisting of glycerol, polyethylene glycol, propylene glycol, polymers having a molecular weight greater than 10000 dalton, pharmaceutical agents, dispersing agents, stabilizers, acids, bases, buffers, antioxidants, emulsifiers, suspending agents, and preservatives; and, (d) the balance water. In some embodiments of the invention, said composition is for topical treatment of skin or mucous membranes in which cell barriers within said skin or mucous membranes are damaged. In some embodiments of the invention, said composition is for topical treatment of skin or mucous membranes in which cell proliferation within said skin or mucous membranes is not in healthy equilibrium with cell differentiation within said skin or mucous membranes. In some embodiments of the invention, said xylitol is present in a concentration of between 2% and 15% (w/w). In some embodiments of the invention, said cholecalciferol is present in a concentration of between 5 μg/ml and 500 μg/ml. In some embodiments of the invention, said glycerol is present in a concentration of between 1% and 15% (w/w). In some preferred embodiments of the invention, said glycerol is present in a concentration of between 2% and 15% (w/w). In some embodiments of the invention, said xylitol is present as an aqueous solution, said aqueous solution being isotonic. In some embodiments of the invention, said xylitol and glycerol are present as an aqueous solution, said aqueous solution of xylitol and glycerol being isotonic.

It is a further object of this invention to disclose a composition as defined in any of the above, wherein said composition comprises less than 0.01% inorganic salt (w/w).

It is a further object of this invention to disclose a composition as defined in any of the above, wherein said composition comprises between 5 μg/ml and 500 μg/ml cholecalciferol.

It is a further object of this invention to disclose a composition as defined in any of the above, wherein said composition includes a polymer having a molecular weight greater than 10000 dalton. In some embodiments of the invention, said polymer is anionic. In some embodiments of the invention, said polymer is selected from the group consisting of carbomer and hyaluronic acid.

It is a further object of this invention to disclose a composition as defined in any of the above, wherein said composition includes a pharmaceutical agent. In some embodiments of the invention, said pharmaceutical agent is a corticosteroid. In some preferred embodiments of the invention, said corticosteroid is hydrocortisone. In some especially preferred embodiments of the invention, the concentration of said hydrocortisone is between 0.5% and 3% (w/w).

It is a further object of this invention to disclose the composition as defined in any of the above, wherein said composition includes at least one auxiliary agent selected from the group consisting of polyethylene glycol, propylene glycol, stabilizers, buffers, antioxidants, emulsifiers, dispersing agents, suspending agents, and preservatives. In some embodiments of the invention, the composition includes a preservative selected from the group consisting of ethylhexylglycerin, phenoxyethanol, and combinations thereof. In some embodiments of the invention, the composition includes a dispersing agent comprising a C10-30 alkyl acrylate crosspolymer.

It is a further object of this invention to disclose the composition as defined in any of the above, wherein said composition includes at least one preservative. In some embodiments of the invention, said preservative is selected from the group consisting of ethylhexylglycerin, phenoxyethanol, and combinations thereof.

It is a further object of this invention to disclose the composition as defined in any of the above, wherein said composition includes at least one dispersing agent. In some embodiments of the invention, said dispersing agent comprises a C10-30 alkyl acrylate crosspolymer.

It is a further object of this invention to disclose the composition as defined in any of the above, wherein said composition is for delivering cholecalciferol to skin via topical application. In some embodiments in which said composition is for delivering cholecalciferol to skin via topical application, the composition is for topical treatment of skin in which said skin is damaged. In some embodiments in which said composition is for delivering cholecalciferol to skin via topical application, the composition is for topical treatment of skin in which cell proliferation within said skin is not in healthy equilibrium with cell differentiation within said skin. In some preferred embodiments of the invention in which the composition is for topical treatment of skin, xylitol and glycerol are each present in a concentration of 5% (w/w).

It is a further object of this invention to disclose the composition as defined in any of the above, wherein said composition is for delivering cholecalciferol to mucous membranes via topical application. In some embodiments in which said composition is for delivering cholecalciferol to mucous membranes via topical application, the composition is for topical treatment of mucous membranes in which said mucous membrane is damaged. In some embodiments in which said composition is for delivering cholecalciferol to mucous membranes via topical application, the composition is for topical treatment of mucous membranes in which cell proliferation within said mucous membranes is not in healthy equilibrium with cell differentiation within said mucous membranes. In some preferred embodiments in which the composition is for delivering cholecalciferol to mucous membranes, the composition comprises an aqueous solution of glycerol and xylitol wherein said aqueous solution of glycerol and xylitol is isotonic. In some preferred embodiments in which the composition is for delivering cholecalciferol to mucous membranes, the composition contains 3% xylitol (w/w) and 1% glycerol (w/w).

It is within the scope of the invention wherein said composition consists of: 5% (w/w) xylitol; 5% (w/w) glycerol; an emulsion of cholecalciferol in an amount to provide 10 μg/ml cholecalciferol; 5% (w/w) propylene glycol; 0.3% (w/w) carbomer; 0.05% (w/w) C10-30 alkyl acrylate crosspolymer; 0.5% (w/w) preservative; sufficient base to adjust the pH to about 5; and the balance water.

It is within the scope of the invention wherein said composition consists of: 3% (w/w) xylitol; 1% (w/w) glycerol; an emulsion of cholecalciferol in oil in an amount to provide 10 μg/ml cholecalciferol; 0.05% (w/w) acrylate/C10-30 crosspolymer; 0.01% (w/w) preservative; sufficient base to adjust the pH to about 6; and the balance water. In some preferred embodiments of the invention, this formulation is provided in the form of a nasal spray.

It is within the scope of the invention wherein said composition consists of: 5% (w/w) xylitol; 5% (w/w) glycerol; an emulsion of cholecalciferol in an amount to provide 50 μg/ml cholecalciferol; 2.5% (w/w) hydrocortisone in oil or in suspension; 5% (w/w) propylene glycol; 0.3% (w/w) carbomer; 0.05% (w/w) acrylate/C10-30 crosspolymer; 0.5% (w/w) preservative; sufficient base to adjust the pH to about 5; and the balance water.

It is within the scope of the invention wherein said composition consists of: 5% (w/w) xylitol; 5% (w/w) glycerol; an emulsion of cholecalciferol in oil in an amount to provide 50 μg/ml cholecalciferol; 2.5% (w/w) hydrocortisone in oil or in suspension; 1.5% (w/w) polyethylene glycol; 0.3% (w/w) carbomer; 0.05% (w/w) acrylate/C10-30 crosspolymer; 0.5% (w/w) preservative; sufficient base to adjust the pH to about 5; and the balance water.

In some embodiments of the invention, said preservative is selected from the group consisting of ethylhexyl glycerin, phenoxyethanol, and combinations thereof In some embodiments of the invention, base is NaOH.

It is a further object of this invention to disclose a composition as defined in any of the above, wherein said composition has undergone a process selected from the group consisting of processes adapted to preserve said composition and processes adapted to maintain said composition in a sterile state.

It is a further object of this invention to disclose a composition as defined in any of the above, wherein said composition is free of any added preservative.

It is a further object of this invention to disclose a composition as defined in any of the above, wherein said process comprises storage in a container selected from the group consisting of sterile single-dose containers and multi-dose containers adapted to maintain the sterility of their contents.

It is a further object of the invention to disclose a composition is as defined in any of the above for topical treatment of skin or mucous membranes in which said skin or mucous membrane is damaged. It is also within the scope of the invention wherein said composition is for topical treatment of skin or mucous membranes in which cell proliferation within said skin or mucous membranes is not in healthy equilibrium with cell differentiation within said skin or mucous membranes.

It is a further object of this invention to disclose a method of preparing a composition for delivering cholecalciferol to skin and/or mucous membranes via topical application, comprising: (a) preparing a first aqueous solution comprising a predetermined amount of a polymer having a molecular weight greater than 10000 dalton; (b) preparing a second aqueous solution comprising predetermined amounts of xylitol and an oil emulsion of cholecalciferol; (c) preparing a third aqueous solution, the components of which comprise said first solution and said second solution, by combining said components under constant mixing; and (d) adjusting a pH of said third aqueous solution under constant mixing.

It is a further object of this invention to disclose such a method, wherein said predetermined amount of a polymer having a molecular weight greater than 10000 dalton is an amount necessary such that said polymer is present in said third solution in a concentration of 0.3% (w/w). In some embodiments of the method, said polymer is anionic. In some embodiments of the method, said polymer is selected from the group consisting of carbomer and hyaluronic acid.

It is a further object of this invention to disclose such a method as defined in any of the above, wherein said predetermined amount of xylitol is an amount such that said third solution has a xylitol concentration of between 2% and 15% (w/w). In some preferred embodiments of the method, said predetermined amount of xylitol is an amount such that said third solution has a xylitol concentration of 5% (w/w). In some preferred embodiments of the method, said predetermined amount of xylitol is an amount such that said third solution is isotonic.

It is a further object of this invention to disclose such a method as defined in any of the above, wherein said predetermined amount of oil emulsion of cholecalciferol is an amount necessary to bring the concentration of cholecalciferol in said third solution to between 5 μg/ml and 500 μg/ml. In some preferred embodiments, said predetermined amount of oil emulsion of cholecalciferol in oil is an amount such that said third solution has a cholecalciferol concentration of between 10 μg/ml and 50 μg/ml.

It is a further object of this invention to disclose the method as defined in any of the above, wherein said step of preparing a second aqueous solution comprises preparing a second aqueous solution comprising predetermined amounts of xylitol, glycerol, and an oil emulsion of cholecalciferol. In some embodiments of the invention, said predetermined amount of glycerol is an amount such that said third solution has a total (glycerol +xylitol) concentration of between 2% and 15% (w/w). In some embodiments of the invention, said predetermined amount of glycerol is an amount such that said third solution has a total (glycerol+xylitol) concentration of 5% (w/w). In some embodiments of the invention, said predetermined amount of glycerol is an amount such that said third solution has a glycerol concentration of about 3% and a total (glycerol+xylitol) concentration of about 4% (w/w). In some embodiments of the invention, said predetermined amounts of xylitol and glycerol are amounts such that said third solution is isotonic.

It is a further object of this invention to disclose the method as defined in any of the above, wherein said step of preparing a first aqueous solution comprises preparing a first aqueous solution comprising a predetermined amount of at least one polymer having a molecular weight greater than 10000 dalton and at least one substance selected from the group consisting of glycerol, pharmaceutical agents, stabilizers, acids, bases, buffers, antioxidants, emulsifiers, suspending agents, and preservatives.

It is a further object of this invention to disclose the method as defined in any of the above, wherein said step of preparing a second aqueous solution comprises preparing a second aqueous solution comprising predetermined amounts of glycerol, an oil emulsion of cholecalciferol, and at least one substance selected from the group consisting of glycerol, pharmaceutical agents, stabilizers, acids, bases, buffers, antioxidants, emulsifiers, suspending agents, and preservatives.

It is a further object of this invention to disclose the method as defined in any of the above, additionally comprising preparing at least one additional aqueous solution comprising at least one substance selected from the group consisting of glycerol, pharmaceutical agents, stabilizers, acids, bases, buffers, antioxidants, emulsifiers, suspending agents, and preservatives, wherein said step of preparing said third aqueous solution comprises preparing a third aqueous solution, the components of which comprise said first solution, said second solution, and said at least one additional aqueous solution, by combining said components under constant mixing.

It is a further object of this invention to disclose the method as defined in any of the above, comprising a step of preserving said composition and/or maintaining said composition in a sterile state by a preservation method that does not comprise any step comprising adding a preservative substance. In some embodiments of the invention, said preservation method comprises storing said composition in a container selected from the group consisting of sterile single-dose containers and multi-dose containers adapted to preserved the sterility of their contents.

It is a further object of this invention to disclose the method as defined in any of the above, consisting of: (a) preparing a first aqueous solution comprising a predetermined amount of a polymer having a molecular weight greater than 10000 dalton; (b) preparing a second aqueous solution comprising predetermined amounts of xylitol, an oil emulsion of cholecalciferol, and optionally at least one substance selected from the group consisting of glycerol, pharmaceutical agents, stabilizers, acids, bases, buffers, antioxidants, emulsifiers, suspending agents, and preservatives; (c) optionally, preparing at least one additional aqueous solution comprising at least one substance selected from the group consisting of glycerol, pharmaceutical agents, stabilizers, acids, bases, buffers, antioxidants, emulsifiers, suspending agents, and preservatives; (d) preparing a third aqueous solution, the components of which comprise said first aqueous solution, said second aqueous solution, and any additional aqueous solutions, by combining said components under constant mixing; (e) optionally, preserving said third aqueous solution and/or maintaining said third aqueous solution in a sterile state by a preservation method that does not comprise any step of adding a preservative substance; and, (f) adjusting a pH of said third aqueous solution under constant mixing. In some embodiments of the method, said predetermined amount of a polymer having a molecular weight greater than 10000 dalton is the amount necessary to bring the concentration of polymer in said third solution to 0.3% (w/w).

In some embodiments of the method, said polymer having a molecular weight greater than 10000 dalton is anionic. In some embodiments of the method, said polymer is selected from the group consisting of carbomer and hyaluronic acid. In some embodiments of the method, said predetermined amount of xylitol is an amount such that said third solution has a xylitol concentration of between 2% and 15% (w/w). In some embodiments of the method, said predetermined amount of xylitol is an amount such that said third solution has a xylitol concentration of 5% (w/w). In some embodiments of the method, said predetermined amount of xylitol is an amount such that said third solution is isotonic.

In some embodiments of the method, said predetermined amount of oil emulsion of cholecalciferol is an amount necessary to bring the concentration of cholecalciferol in said third solution to between 5 μg/ml and 500 μg/ml. In some embodiments of the method, said predetermined amount of oil emulsion of cholecalciferol in oil is an amount such that said third solution has a cholecalciferol concentration of between 10 μg/ml and 50 μg/ml. In some embodiments of the method, said step of preparing a second aqueous solution comprises preparing a second aqueous solution comprising predetermined amounts of xylitol, glycerol, and an oil emulsion of cholecalciferol.

In some preferred embodiments of the method, said third solution is characterized by a xylitol concentration X and a glycerol concentration G, and X+G is between 2% and 15% (w/w). In some preferred embodiments of the method, X+G is 5% (w/w). In some preferred embodiments of the method, X is about 3% (w/w) and X+G is about 4% (w/w). In some preferred embodiments of the method, said predetermined amounts of xylitol and glycerol are amounts such that said third solution is isotonic.

In some preferred embodiments of the method, said step of preparing a first aqueous solution comprises preparing a first aqueous solution comprising a predetermined amount of at least one polymer having a molecular weight greater than 10000 dalton and at least one substance selected from the group consisting of glycerol, pharmaceutical agents, stabilizers, acids, bases, buffers, antioxidants, emulsifiers, suspending agents, and preservatives.

In some preferred embodiments of the method, said step of preparing a second aqueous solution comprises preparing a second aqueous solution comprising predetermined amounts of glycerol, an oil emulsion of cholecalciferol, and at least one substance selected from the group consisting of glycerol, pharmaceutical agents, stabilizers, acids, bases, buffers, antioxidants, emulsifiers, suspending agents, and preservatives.

In some preferred embodiments of the method, said preservation method comprises storing said composition in a container selected from the group consisting of sterile single-dose containers and multi-dose containers adapted to preserved the sterility of their contents. In some preferred embodiments of the method, said preservation method comprises storing said composition in a container selected from the group consisting of sterile single-dose containers and multi-dose containers adapted to preserved the sterility of their contents.

It is a further object of this invention to disclose the use of a composition as defined in any of the above in a therapeutic or cosmetic composition for topical treatment of skin or mucous membranes in which said skin or mucous membrane is damaged. In some embodiments, said use is in a therapeutic composition for topical treatment of skin. In some embodiments, said use is in a therapeutic composition for topical treatment of mucous membranes. In some embodiments, said use is in a cosmetic composition for topical treatment of skin. In some embodiments, said use is in a cosmetic composition for topical treatment of mucous membranes.

It is a further object of this invention to disclose the use of a composition as defined in any of the above in a therapeutic or cosmetic composition for topical treatment of skin or mucous membranes in which cell proliferation within said skin or mucous membranes is not in healthy equilibrium with cell differentiation within said skin or mucous membranes. In some embodiments, said use is in a therapeutic composition for topical treatment of skin. In some embodiments, said use is in a therapeutic composition for topical treatment of mucous membranes. In some embodiments, said use is in a cosmetic composition for topical treatment of skin. In some embodiments, said use is in a cosmetic composition for topical treatment of mucous membranes.

It is a further object of this invention to disclose a method for delivering cholecalciferol to skin or mucous membranes comprising applying topically the composition as defined in any of the above. In some embodiments of the invention, the method is a method for treatment of a condition in which cell barriers within said skin or mucous membranes are damaged. In some embodiments of the invention, the method is a method for treatment of a condition in which cell proliferation within said skin or mucous membranes is not in healthy equilibrium with cell differentiation within said skin or mucous membranes.

### DETAILED DESCRIPTION OF THE PREFERRED EMBODIMENTS

In the following description, various aspects of the invention will be described. For purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is described in the specification, but only as indicated in the accompanying claims, with the proper scope determined only by the broadest interpretation of said claims.

Unless otherwise indicated, all concentrations are expressed as w/w. With reference to numeric quantities, the term “about” refers to a range of ±20% of the nominal value.

The present invention was motivated by the inventor's surprising discovery, based on cell culture studies, that combinations of xylitol demonstrate a synergistic reduction in certain inflammatory mediators. Without being bound by theory, the present invention is also based on the hypothesis that locally applied cholecalciferol adds to the efficiency of the curative effect of sunlight on certain conditions of the skin or mucous membranes, particularly those in which cell barriers within said skin or mucous membranes are damaged or in which cell proliferation within said skin or mucous membranes is not in healthy equilibrium with cell differentiation within said skin or mucous membranes.

In some embodiments of the invention, the composition comprises xylitol and a cholecalciferol emulsion in water. In preferred embodiments of the invention, the concentration of xylitol is between 2% and 15% w/w. In the most preferred embodiments of the invention, the concentration of xylitol is 5% w/w. In the most preferred embodiments of the invention, the composition comprises less than 0.01% inorganic salt. In preferred embodiments of the invention, the composition comprises xylitol in the form of an aqueous xylitol solution.

In some embodiments of the invention, the composition additionally comprises glycerol. In some preferred embodiments of the invention, the concentration of glycerol is between 1% and 15% w/w. In some preferred embodiments of the invention, the concentration of glycerol is between 2% and 15% w/w. In some more preferred embodiments of the invention, the concentration of glycerol is 5% w/w. In some especially preferred embodiments in which the composition is for topical application on the skin, the concentrations of xylitol and glycerol are 5% w/w each. In some especially preferred embodiments of the invention in which the composition is for topical application to mucous membranes, the concentration of xylitol is about 3% w/w and the concentration of glycerol is about 1% w/w. In the most preferred embodiments of the invention in which the composition is for topical application to mucous membranes, the xylitol or xylitol+glycerol solution is isotonic.

In preferred embodiments of the invention, the concentration of cholecalciferol is between 5 μg/ml and 500 μg/ml. In more preferred embodiments of the invention, the concentration of cholecalciferol is between 10 μg/ml and 50 μg/ml.

In some embodiments of the invention, it comprises a polymer having a molecular weight over 10000 dalton. In some preferred embodiments of the invention, the polymer is anionic, for example, carbomer or hyaluronic acid. In some more preferred embodiments of the invention, the polymer is present in a concentration of 0.3%.

The composition may also contain at least one pharmaceutical agent. In some embodiments of the invention, the pharmaceutical agent is a corticosteroid. In some preferred embodiments of the invention, the corticosteroid is hydrocortisone. In some more preferred embodiments of the invention, the hydrocortisone is present in a concentration of 0.5% to 3% w/w.

The composition may optionally include auxiliary agents such as polyethylene glycol, propylene glycol, stabilizers, buffers, acids, bases, antioxidants, emulsifiers, dispersing agents, suspending agents, or preservatives.

Non-limiting examples of preservatives that may be incorporated into the composition herein disclosed include ethylhexylglycerin, phenoxyethanol (typically present in amounts of up to 0.5%). The composition may also be preserved or its sterility maintained by any other suitable method known in the art rather than by addition of one or more preservatives. A non-limiting example of such a method is storage in a sealed sterile single-dose container or special sterile multi-dose container. Storage in such a container will maintain the sterility of the composition until it is used.

Non-limiting examples of dispersing agents include acrylates such as C10-30 alkyl acetate cross polymer. In some embodiments of the invention, the base is added to control the pH of the final product. A non-limiting example of a suitable base is NaOH.

One non-limiting exemplary preferred embodiment of the invention can be prepared by the following method. First, a polymer having a molecular weight over 10000 daltons (in preferred embodiments, carbomer) is mixed with water to form a first solution. Xylitol, cholecalciferol (in the form of an oil emulsion), and any optional components are mixed with water separately to form a second solution. The two solutions are then combined under constant mixing to form a third solution. The concentrations of the components in the first and second solutions are chosen so that when the two solutions are mixed, the concentrations in the third solution will have predetermined values. In preferred embodiments, these predetermined values are as disclosed above or as disclosed in the examples below.

The pH of the third solution is then adjusted. Normally, the pH is adjusted by addition of base to bring the pH to one that is compatible with topical application to skin or mucous membranes. In preferred embodiments of the invention in which compatibility with the skin is desired, the pH is adjusted so that it lies within the range of 4.5 to 5.5. In preferred embodiments of the invention in which compatibility with a mucous membrane is desired, the pH is adjusted so that it lies with the range of 4.5 to 7.5, depending on the particular mucous membrane with which compatibility is desired.

It is emphasized that in preferred embodiments of the invention, there are no therapeutically effective components present in therapeutically effective concentrations other than those explicitly recited herein as being part of the composition. In the most preferred embodiments of the invention, no other components whatsoever are added other than those specifically recited herein as being part of the composition, i.e. including components that have no therapeutic or cosmetic effect.

The instant invention also provides a novel method of application of cholecalciferol to skin or mucous membranes. The method comprises applying topically the composition as defined in any of the above to the skin or mucous membrane(s) in need of treatment. The composition is applied to the skin or mucous membrane as needed. In preferred embodiments, the composition is applied topically 2-4 times daily or as advised by a physician or pharmacist. The treatment continues as needed, or as determined by a physician or pharmacist. In some embodiments of the invention, the method is a method for treatment of a condition in which cell barriers within said skin or mucous membranes are damaged. In some embodiments of the invention, the method is a method for treatment of a condition in which cell proliferation within said skin or mucous membranes is not in healthy equilibrium with cell differentiation within said skin or mucous membranes.

In order to illustrate the invention and to enable one of ordinary skill in the art to make and use it, a number of non-limiting examples of the composition, its preparation, and its use are now provided.

### EXAMPLE 1

A topical preparation for the treatment of dry, atopic, or psioratic skin was prepared as described above, consisting of:
Xylitol 5% w/w
Glycerol 5% w/w
Cholecalciferol (D-vitamin oil) emulsion in oil 10 μg/ml (˜0.04% w/w)
Ethylhexyglycerin, Phenoxyethanol (preservative) 0.5% w/w
Acrylates/C10-30 Alkyl Acrylate Crosspolymer (dispersing agent) 0.05% w/w
Propylene Glycol 5% w/w
Carbomer 0.3% w/w
Balance water

The pH of the composition was adjusted to about 5 by addition of NaOH.

### EXAMPLE 2

A nose spray to treat dry or allergic nose mucous membrane was prepared as described above, consisting of:
Xylitol 3% w/w
Glycerol 1% w/w
Cholecalciferol (D-vitamin oil) emulsion in oil 10 μg/ml
Acrylates/C10-30 Alkyl Acrylate Crosspolymer (dispersing agent) 0.05% w/w
Preservative 0.01%
Balance water

The pH of the composition was adjusted to about 6 by addition of NaOH.

### EXAMPLE 3

A topical preparation containing cortisone was prepared as described above, consisting of:
Xylitol 5% w/w
Glycerol 5% w/w
Cholecalciferol (D-vitamin oil) emulsion 50 μg/ml
Hydrocortisone 2.5% w/w in oil or in suspension
Propylene glycol 5% w/w
Acrylates/C10-30 Alkyl Acrylate Crosspolymer (dispersing agent) 0.05% w/w
Carbomer (Ultrez 10) 0.3% w/w
Preservative 0.5%
Balance water

The pH of the composition was adjusted to about 5 by addition of NaOH.

An alternative formulation for the topical preparation containing cortisone was prepared with the same components as those listed above, except that instead of 5% w/w propylene glycol, 1.5% w/w polyethylene glycol was used.

### EXAMPLE 4

A clinical study was performed. The composition as disclosed in example 1 above was given to 6 patients, who were given for comparison the composition as disclosed in example 1 except that it did not contain any cholecalciferol. The patients greatly preferred the composition containing the cholecalciferol.

1-126. (canceled)

127. A composition for use in a treatment of skin by topical delivery of cholecalciferol to said skin, said skin characterized by a symptom selected from the group consisting of:
cell barriers within said skin are damaged; and,
cell proliferation within said skin is not in healthy equilibrium with cell differentiation within said skin;
wherein said composition consists of:
xylitol;
cholecalciferol in emulsion;
optionally, at least one substance selected from the group consisting of glycerol, polyethylene glycol, propylene glycol, polymers having a molecular weight greater than 10000 dalton, pharmaceutical agents, dispersing agents, stabilizers, acids, bases, buffers, antioxidants, emulsifiers, suspending agents, and preservatives; and,
the balance water.

128. The composition according to claim 127, wherein said composition consists of an emulsion consisting of:
a dispersed phase comprising cholecalciferol; and,
an aqueous phase consisting of:
xylitol; and,
optionally, at least one substance selected from the group consisting of glycerol, polyethylene glycol, propylene glycol, polymers having a molecular weight greater than 10000 dalton, pharmaceutical agents, dispersing agents, stabilizers, acids, bases, buffers, antioxidants, emulsifiers, suspending agents, and preservatives.

129. The composition according to claim 128, wherein said dispersed phase consists of cholecalciferol in oil.

130. The composition according to claim 127, wherein at least one of the following is true:
said composition comprises less than 0.01% inorganic salt (w/w);
said cholecalciferol is present in a concentration of between 5 μg/ml and 500 μg/ml;
said pharmaceutical agent is hydrocortisone and said hydrocortisone is present in a concentration of between 0.5% and 3% (w/w);
said xylitol is in aqueous solution, and further wherein said aqueous solution of xylitol is isotonic;
said xylitol is present in a concentration of between 2% and 15% (w/w);
said glycerol is present in a concentration of between 1% and 15% (w/w); and,
said base is NaOH.

131. The composition according to claim 127, wherein said xylitol is present in a concentration of 5% (w/w).

132. The composition according to claim 127, wherein said composition comprises 5% glycerol (w/w) and 5% xylitol (w/w).

133. The composition according to claim 132, wherein said xylitol and glycerol are in aqueous solution, and further wherein said aqueous solution of xylitol and glycerol is isotonic.

134. The composition according to claim 127, wherein said polymer is selected from the group consisting of carbomer and hyaluronic acid.

135. The composition according to claim 127, wherein said pharmaceutical agent is hydrocortisone and said hydrocortisone is present in a concentration of between 0.5% and 3% (w/w).

136. The composition according to claim 127, wherein said preservative is selected from the group consisting of ethylhexylglycerin, phenoxyethanol, and combinations thereof.

137. The composition according to claim 127, wherein said dispersing agent comprises a C10-30 alkyl acrylate crosspolymer.

138. The composition according to claim 127 wherein said composition has undergone a process selected from the group consisting of processes adapted to preserve said composition and processes adapted to maintain said composition in a sterile state.

139. The composition according to claim 127, wherein said composition is free of any added preservative.

140. The composition according to claim 127, consisting of:
5% (w/w) xylitol;
5% (w/w) glycerol;
an emulsion of cholecalciferol in an amount to provide 10 μg/ml cholecalciferol;
5% (w/w) propylene glycol;
0.3% (w/w) carbomer;
0.05% (w/w) C10-30 alkyl acrylate crosspolymer;
0.5% (w/w) preservative;
sufficient base to adjust the pH to about 5; and,
the balance water.

141. The composition according to claim 127, consisting of:
5% (w/w) xylitol;
5% (w/w) glycerol;
an emulsion of cholecalciferol in an amount to provide 50 μg/ml cholecalciferol;
2.5% (w/w) hydrocortisone in oil or in suspension;
0.3% (w/w) carbomer;
0.05% (w/w) acrylate/C10-30 crosspolymer;
0.5% (w/w) preservative;
either:
5% (w/w) propylene glycol;
or:
1.5% (w/w) propylene glycol;
sufficient base to adjust the pH to about 5; and,
the balance water.

142. A method of preparing a composition for topical use in a treatment of skin characterized by a symptom selected from the group consisting of:
cell barriers within said skin are damaged; and,
cell proliferation within said skin is not in healthy equilibrium with cell differentiation within said skin;
wherein said method comprises:
preparing a first aqueous solution comprising a predetermined amount of a polymer having a molecular weight greater than 10000 dalton and optionally at least one substance selected from the group consisting of glycerol, pharmaceutical agents, stabilizers, acids, bases, buffers, antioxidants, emulsifiers, suspending agents, and preservatives;
preparing a second aqueous solution comprising predetermined amounts of xylitol, an oil emulsion of cholecalciferol, and optionally at least one substance selected from the group consisting of glycerol, pharmaceutical agents, stabilizers, acids, bases, buffers, antioxidants, emulsifiers, suspending agents, and preservatives;
either:
preparing a third aqueous solution, the components of which comprise said first solution and said second solution, by combining said components under constant mixing;
or:
preparing at least one additional aqueous solution comprising at least one substance selected from the group consisting of glycerol, pharmaceutical agents, stabilizers, acids, bases, buffers, antioxidants, emulsifiers, suspending agents, and preservatives; and,
preparing a third aqueous solution, the components of which comprise said first solution, said second solution, and said at least one additional aqueous solution, by combining said components under constant mixing;
and, adjusting a pH of said third aqueous solution under constant mixing.

143. The method according to claim 142, wherein at least one of the following is true:
said predetermined amount of a polymer having a molecular weight greater than 10000 dalton is an amount such that said third solution has a polymer concentration of 0.3% (w/w);
said predetermined amount of xylitol is an amount such that said third aqueous solution has a xylitol concentration of between 2% and 15% (w/w);
said third solution is characterized by a xylitol concentration X and a glycerol concentration G, and further wherein X+G is between 2% and 15% (w/w);
said third solution is characterized by a xylitol concentration X and a glycerol concentration G, and further wherein X+G is 5% (w/w);
said third solution is isotonic; and,
said predetermined amount of oil emulsion of cholecalciferol is an amount such that said third solution has a cholecalciferol concentration of between 5 μg/ml and 500 μg/ml.

144. A method for delivering cholecalciferol to skin, comprising applying topically the composition according to claim 127.

145. The method according to claim 144, comprising treating a condition in which said skin is damaged by applying topically the composition according to claim 127.

146. The method according to claim 144, comprising treating a condition in which cell proliferation within said skin is not in healthy equilibrium with cell differentiation within said skin by applying topically the composition according to claim 127.
